(19) United States
(12) Patent Application Publication
(10) Pub. No.: US 2021/0295151 A1
(43) Pub. Date: Sep. 23, 2021

(54) METHOD OF MACHINE-LEARNING BY COLLECTING FEATURES OF DATA AND APPARATUS THEREOF

(71) Applicant: Lunit Inc., Seoul (KR)

(72) Inventors: Donggeun YOO, Seoul (KR); Jeong Hoon LEE, Seoul (KR); Jae Hwan LEE, Seoul (KR)

(21) Appl. No.: 17/077,114

(22) Filed: Oct. 22, 2020

(30) Foreign Application Priority Data
Mar. 20, 2020 (KR) 10-2020-0034456

(51) Int. Cl. G06N 3/08; G06N 3/04; G06K 9/62; G06K 9/46

(52) U.S. Cl. CPC G06N 3/08; G06K 9/46; G06K 9/6262; G06N 3/04

(57) ABSTRACT

There is provided a method and apparatus that collects feature points of data and performs machine learning. A machine learning method comprises receiving first feature data obtained by applying a basic model to first analysis target data, receiving second feature data obtained by applying the basic model to second analysis target data, and obtaining a final machine learning model through performing machine learning on a correlation between the first feature data and first analysis result data and a correlation between the second feature data and second analysis result data.

### CROSS-REFERENCE TO RELATED APPLICATION

This application claims priority to and the benefit of Korean Patent Application No. 10-2020-0034456 filed in the Korean Intellectual Property Office on Mar. 20, 2020, the entire contents of which are incorporated herein by reference.

### BACKGROUND

### (a) Field

The present disclosure relates to a method and apparatus that collects feature points of data and performs machine learning. More specifically, the present disclosure relates to a machine learning method and apparatus for improving the performance thereof while protecting sensitive information related to data.

### (b) Description of the Related Art

In order to obtain the best machine learning model, it is required to perform machine learning based on as much input data as possible. However, in some cases, it is difficult to take out data from a data center due to physical environmental issues or security issues regarding where data is stored. For example, since medical data is classified as sensitive personal information, if target data of a data center is medical data, many restrictions are placed on data sharing. This is because medical data includes sensitive information related to personal information, such as identity of a patient, information on disease from which the patient is suffering, information on disease that the patient is likely to get, or a family history of the patient. Therefore, researches on various methods to improve the performance of machine learning models by using all the data sets of each center have been continued.

For example, a technique called federated learning has recently been in the limelight. This technique is an algorithm that installs an independent deep learning model in each institution, synchronizes with a master server, and makes it possible to train a deep learning model as if using integrated data. This is an algorithm that may have an effect of sharing sensitive data without real sharing thereof.

However, the federated learning requires the cooperation of institutions, in that each of the independent models should be able to access data provided by hospitals and an environment where a trained model can synchronize with a model existing in an external master server should be implemented. Though, medical institutions that focus on protecting personal information generally use an intra-network that makes it possible to share data only within each of medical institutions. Therefore, there are many difficulties in implementing the federated model under the condition of being disconnected from the outside.

### SUMMARY

A machine learning method according to the present disclosure comprises receiving first feature data obtained by applying a basic model to first analysis target data, receiving second feature data obtained by applying the basic model to second analysis target data, and obtaining a final machine learning model through performing machine learning on a correlation between the first feature data and first analysis result data and a correlation between the second feature data and second analysis result data.

The machine learning method according to the present disclosure further comprises receiving predictive feature data obtained by applying the basic model to test data, and obtaining predictive result data corresponding to the test data by applying the final machine learning model to the predictive feature data.

In the machine learning method according to the present disclosure, the first analysis target data and the second analysis target data are related to medical images obtained in different environments.

In the machine learning method according to the present disclosure, the first feature data, the second feature data, and the predictive feature data are obtained by performing lossy compression of the first analysis target data, the second analysis target data, and the test data, respectively.

In the machine learning method according to the present disclosure, the first analysis target data, the second analysis target data and the test data include personal information, and the personal information is not identified in the first feature data, the second feature data, and the predictive feature data.

In the machine learning method according to the present disclosure, the first analysis result data is a result obtained through analyzing the first analysis target data by a user, and the second analysis result data is a result obtained through analyzing the second analysis target data by the user.

In the machine learning method according to the present disclosure, the first feature data is at least one layer of a plurality of first feature layers obtained by applying the first analysis target data to the basic model, and the second feature data is at least one layer of a plurality of second feature layers obtained by applying the second analysis target data to the basic model.

In the machine learning method according to the present disclosure, the first feature data and the second feature data are obtained by selecting a layer located in a same position in the plurality of first feature layers and the plurality of second feature layers, respectively.

In the machine learning method according to the present disclosure, the basic model is a previously learned machine learning model for image classification.

In the machine learning method according to the present disclosure, the basic model is a sub-machine learning model that is obtained through machine learning of a first training data set including the first analysis target data and the first analysis result data.

The machine learning apparatus according to the present disclosure includes a processor a memory. Based on instructions stored in the memory, the processor receives first feature data obtained by applying a basic model to first analysis target data, receives second feature data obtained by applying the basic model to second analysis target data, and obtains a final machine learning model through machine learning of a correlation between the first feature data and first analysis result data and a correlation between the second feature data and second analysis result data.

The processor of the machine learning apparatus according to the present disclosure, based on the instructions stored in the memory, the processor receives predictive feature data obtained by applying a basic model to test data, and obtains predictive result data corresponding to the test data by applying the final machine learning model to the predictive feature data.

In the machine learning apparatus according to the present disclosure, the first analysis target data and the second analysis target data are related to medical images obtained in different environments.

In the machine learning apparatus according to the present disclosure, the first feature data, the second feature data, and the predictive feature data are obtained by lossy compression of the first analysis target data, the second analysis target, data and the test data, respectively.

In the machine learning apparatus according to the present disclosure, the first analysis target data, the second analysis target data, and the test data include personal information, and the personal information is not identified in the first feature data, the second feature data, and the predictive feature data.

In the machine learning apparatus according to the present disclosure, the first analysis result data is a result obtained through analyzing the first analysis target data by a user, and the second analysis result data is a result obtained through analyzing the second analysis target data by the user.

In the machine learning apparatus according to the present disclosure, the first feature data is at least one layer of a plurality of first feature layers obtained by applying the first analysis target data to the basic model, and the second feature data is at least one layer of a plurality of second feature layers obtained by applying the second analysis target data to the basic model.

In the machine learning apparatus according to the present disclosure, the first feature data and the second feature data are obtained by selecting a layer located in the same position in the plurality of first feature layers and the plurality of second feature layers, respectively.

In the machine learning apparatus according to the present disclosure, the basic model is a previously learned machine learning model for image classification.

In the machine learning apparatus according to the present disclosure, the basic model is a sub-machine learning model that is obtained through machine learning of a first training data set including the first analysis target data and the first analysis result data.

In addition, a program that implements an operation method of the above-described machine learning apparatus may be recorded in a computer readable medium.

### BRIEF DESCRIPTION OF THE DRAWINGS

FIG. 1 is a block diagram of a machine learning apparatus 100 according to an embodiment of the present disclosure.

FIG. 2 is a diagram showing a machine learning apparatus according to an embodiment of the present disclosure.

FIG. 3 is a flowchart showing an operation of a machine learning apparatus according to an embodiment of the present disclosure.

FIG. 4 is a diagram showing a process of obtaining an integrated machine learning model according to an embodiment of the present disclosure.

FIG. 5 is a diagram for explaining a process to generate a sub-machine learning model according to an embodiment of the present disclosure.

FIG. 6 is a flowchart showing a method for generating a final machine learning model according to an embodiment of the present disclosure.

FIG. 7 is a diagram for explaining a method for generating a final machine learning model according to an embodiment of the present disclosure.

FIG. 8 is a diagram for explaining feature data according to an embodiment of the present disclosure.

FIG. 9 is a diagram for explaining a process of generating feature data according to an embodiment of the present disclosure.

FIG. 10 is a diagram illustrating a process of generating feature data according to an embodiment of the present disclosure.

FIG. 11 is a diagram showing a process of generating a final machine learning model 710 according to an embodiment of the present disclosure

FIG. 12 is a flowchart showing an operation of a machine learning apparatus according to an embodiment of the present disclosure.

FIG. 13 is a diagram showing an operation of a machine learning apparatus according to an embodiment of the present disclosure.

### DETAILED DESCRIPTION

Advantages and features of the disclosed embodiments, and methods for accomplishing them will become apparent from the following embodiments described with reference to the accompanying drawings. The present disclosure, however, may be embodied in many different forms and should not be construed as being limited to the embodiments set forth herein. Rather, these embodiments are provided so that this disclosure will be thorough and complete and will fully convey the concept of the disclosure to the person of ordinary skill in the art.

Hereinafter, the terms used in the specification will be briefly described and the disclosed embodiments will be described in detail.

As used herein, the terms used in the present disclosure are selected from general terms that are widely used while taking into account the functions of the present disclosure, but these may vary depending on the intention of a person skilled in the art, judicial precedents, or the emergence of new technologies. In addition, in certain cases, there may be a term arbitrarily selected by the applicant, in which case the meaning thereof will be described in the corresponding description. Therefore, it is intended that the terminology used herein should be interpreted based on the meaning of the term and on the entire contents of the specification, rather than on the name of the term.

In this specification, the singular forms “a”, “an” and “the” are intended to include the plural forms as well, unless the context clearly indicates otherwise, and vise versa.

As used herein, unless explicitly described to the contrary, the word “comprise”, “include” or “have”, and variations such as “comprises”, “comprising”, “includes”, “including”, “has” or “having” will be understood to imply the inclusion of stated elements but not the exclusion of any other elements.

In addition, the term “unit” used in the specification refers to a software or hardware component, and the “unit” performs a certain function. However, the “unit” is not limited to a software or hardware. The “unit” may be configured to be in an addressable storage medium or to reproduce one or more processors. Thus, as an example, the “unit” includes components such as software components, object-oriented software components, class components, and task components. Further, the “unit” includes processes, functions, properties, procedures, subroutines, segments of a program code, drivers, firmware, microcode, circuit, data, database, data structures, tables, arrays and variables. Functions provided within the components and “units” may be implemented by combining a smaller number of components and “units”, or by dividing into additional components and “units”.

According to an embodiment of the present disclosure, the “unit” may be implemented with a processor and a memory. The term “processor” should be broadly construed to include a general purpose processor, a central processing unit (CPU), a microprocessor, a digital signal processor (DSP), a controller, a microcontroller, a state machines, and the like. In some environments, the “processor” may refer to an application specific integrated circuit (ASIC), a programmable logic device (PLD), a field programmable gate array (FPGA), and the like. The term “processor” may refer to a combination of processing devices, such as a combination of a DSP and a microprocessor, a combination of a plurality of microprocessors, a combination of at least one of microprocessor coupled with a DSP core, or a combination of any other such components.

The term “memory” should be broadly interpreted to include an electronic component capable of storing electronic information. The term “memory” may refer to various types of processor-readable media, such as a random access memory (RAM), a read only memory (ROM), a non-volatile random access memory (NVRAM), a programmable read only memory (PROM), an erasable programmable ROM (EPROM), and an electrically erasable programmable ROM (EEPROM), a flash memory, a magnetic or optical data storage, registers, and the like. If a processor can read information from a memory and/or records information on the memory, the memory is referred to be in a state of electronic communication with the processor. The memory integrated in a processor is in a state of electronic communication with the processor.

Hereinafter, embodiments of the present disclosure will be described in detail with reference to the attached drawings so that the person of ordinary skill in the art may easily implement the present disclosure. In the drawings, elements irrelevant to the description of the present disclosure are omitted for clarity of the explanation.

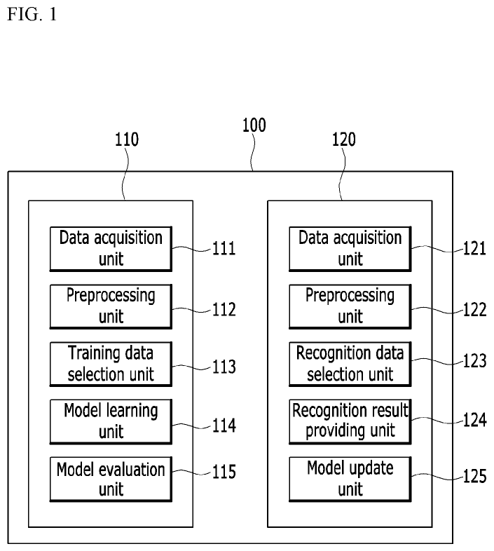

FIG. 1 is a block diagram of a machine learning apparatus 100 according to an embodiment of the present disclosure.

Referring to FIG. 1, the machine learning apparatus 100 according to an embodiment may include at least one of a data learning unit 110 and a data recognition unit 120. The machine learning apparatus 100 as above-described may include a processor and a memory.

The data learning unit 110 may learn a machine learning model for performing a target task by using a data set. The data learning unit 110 may receive a data set and label information related to the target task. The data learning unit 110 may obtain a machine learning model by performing machine learning on a relationship between the data set and the label information. The machine learning model obtained by the data learning unit 110 may be a model for generating label information using a data set.

The data recognition unit 120 may receive and store the machine learning model of the data learning unit 110. The data recognition unit 120 may output label information by applying the machine learning model to input data. Further, the data recognition unit 120 may use input data, the label information, and a result output by the machine learning model for updating the machine learning model.

At least one of the data learning unit 110 and the data recognition unit 120 may be manufactured in the form of at least one hardware chip and mounted on an electronic device. For example, at least one of the data learning unit 110 and the data recognition unit 120 may be manufactured in the form of a dedicated hardware chip for artificial intelligence (AI), or manufactured as a part of an existing general purpose processor (e.g., CPU or application processor) or a graphic-only processor (e.g., GPU), and then may be mounted on the above-described various electronic devices.

In addition, the data learning unit 110 and the data recognition unit 120 may be mounted on separate electronic devices, respectively. For example, one of the data learning unit 110 and the data recognition unit 120 may be included in an electronic device, and the other may be included in a server. In addition, via wired or wireless connection between the data learning unit 110 and the data recognition unit 120, the data learning unit 110 may provide the data recognition unit 120 with information of a machine learning model constructed by the data learning unit 110, and data input into the data recognition unit 120 may be provided to the data learning unit 110 as additional training data.

Meanwhile, at least one of the data learning unit 110 and the data recognition unit 120 may be implemented as a software module. When at least one of the data learning unit 110 and the data recognition unit 120 is implemented as a software module (or a program module including instructions), the software module may be stored in a memory or non-transitory computer readable media. In addition, in this case, at least one software module may be provided by an operating system (OS) or by a predetermined application. Alternatively, part of at least one software module may be provided by an operating system (OS), and the rest may be provided by a predetermined application.

The data learning unit 110 according to an embodiment of the present disclosure may include a data acquisition unit 111, a preprocessing unit 112, a training data selection unit 113, a model learning unit 114, and a model evaluation unit 115.

The data acquisition unit 111 may acquire data required for machine learning. Since a large amount of data is required for learning, the data acquisition unit 111 may receive a data set including multiple data.

Label information may be assigned to each of the multiple data. The label information may be information explaining each of the multiple data. The label information may be information which a target task may aim to derive. The label information may be obtained from user input, a memory, or result of a machine learning model. For example, if the target task is to determine the existence of a specific object from an image, the multiple data may be a plurality of image data and the label information may be whether a specific object exists in each of the plurality of images.

The preprocessing unit 112 may preprocess the acquired data so that the received data is available for machine learning. The preprocessing unit 112 may process the obtained data set into a predetermined format, for the use by a model learning unit 114 to be described later.

The training data selection unit 113 may select data required for learning among the preprocessed data. The selected data may be provided to the model learning unit 114. The training data selection unit 113 may select data required for learning among the preprocessed data according to predetermined criteria. In addition, the training data selection unit 113 may select data according to the criteria predetermined through learning by the model learning unit 114 to be described later.

The model learning unit 114 may learn a criterion regarding which label information is to be output based on the data set. In addition, the model learning unit 114 may perform machine learning by using the data set and label information on the data set as training data. Also, the model learning unit 114 may perform machine learning by additionally using a previously obtained machine learning model. In this case, the previously obtained machine learning model may be a prebuilt model. For example, the machine learning model may be a model built in advance by receiving basic training data.

The machine learning model may be built in consideration of the application field of the learning model, the purpose of learning, the computer performance of a device, and the like. The machine learning model may be a model based on, for example, a neural network. For example, models such as a deep neural network (DNN), a recurrent neural network (RNN), a long short-term memory model (LSTM), a bidirectional recurrent deep neural network (BRDNN), and a convolutional neural network (CNN) may be used as a machine learning model, but not limited thereto.

According to various embodiments, when a plurality of pre-built machine learning models are provided, the model learning unit 114 may determine a machine learning model whose basic training data is highly related to the input training data, as a machine learning model to be learned. In this case, the basic training data may be pre-classified for each type of data and also the machine learning model may be pre-built for each data type. For example, the basic training data may be pre-classified by various criteria such as a place where the training data was generated, time when the training data was generated, the size of the training data, a generator of the training data, and a type of object in the training data.

In addition, the model learning unit 114 may learn a machine learning model by using a learning algorithm including, for example, error back-propagation or gradient descent.

Additionally, the model learning unit 114 may learn a machine learning model through, for example, supervised learning using the training data as input values. In addition, the model learning unit 114 may obtain a machine learning model, for example, through unsupervised learning to discover criteria for a target task by performing self-learning on the data type required for the target task without any supervision. Further, the model learning unit 114 may learn the machine learning model through reinforcement learning that uses feedback on whether the result of the target task according to the learning is correct or not.

In addition, when the machine learning model is learned, the model learning unit 114 may store the trained machine learning model. In this case, the model learning unit 114 may store the trained machine learning model in a memory of the electronic device including the data recognition unit 120. Alternatively, the model learning unit 114 may store the learned machine learning model in a memory of a server connected with an electronic device via a wired or wireless network.

The memory where the learned machine learning model is stored may also store, for example, instructions or data related to at least one of other components of the electronic device. Also, the memory may store software and/or a program. The program may include, for example, a kernel, a middleware, an application programming interface (API) and/or an application program (or “application”).

The model evaluation unit 115 may input evaluation data to the machine learning model, and may make the machine learning unit 114 relearn when a result output from the evaluation data does not satisfy predetermined critera. In this case, the evaluation data may be preset data for evaluating the machine learning model.

For example, it may be determined that the predetermined criteria is not met, when the number or ratio of the evaluation data whose recognition result is not correct is larger than a predetermined threshold, among the result of the learned machine learning model for the evaluation data. For instance, the predetermined criteria may be defined as the ratio of 2%. In this case, if the learned machine learning model outputs incorrect recognition results for the evaluation data larger than 20 of the total 1000 evaluation data, the model evaluation unit 115 may determine that the trained machine learning model is not appropriate.

Meanwhile, when there is a plurality of learned machine learning models, the model evaluation unit 115 may evaluate whether each of leraned trained machine learning models satisfies the predetermined criteria and may determine a model satisfying the predetermined criteria as a final machine learning model. In this case, when it is evaluated that a plurality of models satisfies the predetermined criteria, the model evaluation unit 115 may determine any one or a predetermined number of models as the final machine learning model, according to the order of the evaluation score.

Meanwhile, at least one of the data acquisition unit 111, the preprocessing unit 112, the training data selection unit 113, the model learning unit 114 and the model evaluation unit 115 in the data learning unit 110 may be manufactured in the form of at least one hardware chip and mounted on an electronic device. For example, at least one of the data acquisition unit 111, the preprocessing unit 112, the training data selection unit 113, the model learning unit 114, and the model evaluation unit 115 may be manufactured in the form of a dedicated hardware chip for an artificial intelligence (AI) or as a part of an existing general processor (e.g., CPI or application processor) and may be mounted on the above-described various electronic devices.

In addition, the data acquisition unit 111, the preprocessing unit 112, the training data selection unit 113, the model learning unit 114, and the model evaluation unit 115 may be mounted on one electronic device or may be mounted on separate electronic devices respectively. For example, some of the data acquisition unit 111, the preprocessing unit 112, the training data selection unit 113, the model learning unit 114, and the model evaluation unit 115 may be included in the electronic device and the rest may be included in the server.

In addition, at least one of the data acquisition unit 111, the preprocessing unit 112, the training data selection unit 113, the model learning unit 114, and the model evaluation unit 115 may be implemented as a software module. If at least one of the data acquisition unit 111, the preprocessing unit 112, the training data selection unit 113, the model learning unit 114, and the model evaluation unit 115 is implemented as a software module (or a program module including instructions), the software module may be stored in non-transitory computer readable media. In addition, in this case, at least one software module may be provided by an operating system (OS) or by a predetermined application. Alternatively, part of the at least one software module may be provided by an operating system (OS), and the rest may be provided by a predetermined application.

The data recognition unit 120 according to an embodiment of the present disclosure may include a data acquisition unit 121, a preprocessing unit 122, a recognition data selection unit 123, a recognition result providing unit 124, and a model update unit 125.

The data acquisition unit 121 may receive input data. The preprocessing unit 122 may preprocess the acquired input data so as to be used by the recognition data selection unit 123 or the recognition result providing unit 124.

The recognition data selection unit 123 may select the necessary data from the preprocessed data. The selected data may be provided to the recognition result providing unit 124. The recognition data selection unit 123 may select some or all of the preprocessed data according to predetermined criteria. In addition, the recognition data selection unit 123 may select data according to the criteria predetermined through learning by the model learning unit 114.

The recognition result providing unit 124 may obtain result data by applying the selected data to the machine learning model. The machine learning model may be a machine learning model generated by the model learning unit 114. The recognition result providing unit 124 may output result data.

The model update unit 125 may update the machine learning model based on the evaluation of the recognition result provided by the recognition result providing unit 124. For example, the model update unit 125 may provide the recognition result provided by the recognition result providing unit 124 to the model learning unit 114, so that the model learning unit 114 may update the machine learning model.

Meanwhile, at least one of the data acquisition unit 121, the preprocessing unit 122, the recognition data selection unit 123, the recognition result providing unit 124, and the model update unit 125 in the data recognition unit 120 may be manufactured in the form of at least one hardware chip and may be mounted on the electronic device. For example, at least one of the data acquisition unit 121, the preprocessing unit 122, the recognition data selection unit 123, the recognition result providing unit 124, and the model update unit 125 may be manufactured in the form of a dedicated hardware chip for artificial intelligence (AI), or as a part of an existing general purpose processor (e.g., CPU or application processor) or a graphic-only processor (e.g., GPU), and may be mounted on the above-described various electronic devices.

In addition, the data acquisition unit 121, the preprocessing unit 122, the recognition data selection unit 123, the recognition result providing unit 124, and the model update unit 125 may be mounted on one electronic device or on separate electronic devices respectively. For example, some of the data acquisition unit 121, the preprocessing unit 122, the recognition data selection unit 123, the recognition result providing unit 124, and the model update unit 125 may be included in the electronic device, and the rest may be included in the server.

In addition, at least one of the data acquisition unit 121, the preprocessing unit 122, the recognition data selection unit 123, the recognition result providing unit 124, and the model update unit 125 may be implemented as a software module. When at least one of the data acquisition unit 121, the preprocessing unit 122, the recognition data selection unit 123, the recognition result proving unit 124, and the model update unit 125 is implemented as a software module (or a program module including instructions), the software module may be stored in non-transitory computer readable media. In addition, in this case, at least one software module may be provided by an operating system (OS) or by a predetermined application. Otherwise, part of the at least one software module may be provided by an operating system (OS), and the rest may be provided by a predetermined application.

Hereinafter, a method and apparatus for a data learning unit 110 to sequentially perform machine learning on data sets will be described in detail.

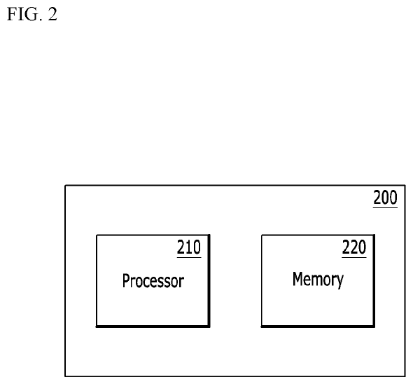

FIG. 2 is a diagram showing a machine learning apparatus according to an embodiment of the present disclosure.

The machine learning apparatus 200 may include a processor 210 and a memory 220. The processor 210 may execute instructions stored in the memory 220.

As described above, the machine learning apparatus 200 may include at least one of the data learning unit 110 and the data recognition unit 120. At least one of the data learning unit 110 or the data recognition unit 120 may be implemented by the processor 210 and the memory 220.

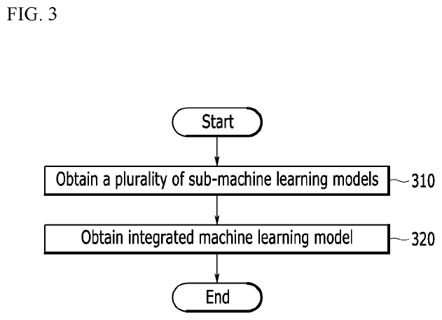

FIG. 3 is a flowchart showing an operation of a machine learning apparatus according to an embodiment of the present disclosure.

The machine learning apparatus 200 may perform step 310 of receiving a plurality of sub-machine learning models obtained by performing machine learning on a plurality of independent training data sets, respectively. The training data set may include analysis target data and analysis result data. The analysis target data may include at least one of medical images of a patient and various measurement data regarding the patient. The analysis target data may be sensitive data because personal information is included therein. Thus, the analysis target data may not be shared without restraint.

The analysis result data may represent a diagnosis result made for the analysis target data by a medical professional. For example, first analysis result data may be a result obtained through analyzing a first analysis target data by a user and a second analysis result data may be a result obtained through analyzing a second analysis target data by the user.

A plurality of sub-machine learning models may be acquired by using at least one of a deep neural network (DNN), a recurrent neural network (RNN), a long short-term memory model (LSTM), a bidirectional recurrent deep neural network (BRDNN), and a convolutional neural network. All the plurality of sub-machine learning models may be obtained using the same algorithm.

Each of the plurality of sub-machine learning models may be executed in different data centers. The plurality of sub-machine learning models may be generated by using the analysis target data included in the training data set and the analysis result data corresponding to a label. Each of the plurality of sub-machine learning models may include at least one weight. At least one weight may be applied to the analysis target data and used to generate a prediction label. At least one weight included in the plurality of sub-machine learning models may be updated through calculations included in forward propagation and back propagation. Further, at least one optimal weight for inferring a label to be predicted from test data may be obtained. Sensitive information, for example, personal information included in a plurality of training data sets may not be backtraced from at least one weight included in the plurality of sub-machine learning models. Accordingly, the machine learning apparatus 200 residing outside a data center may receive the plurality of sub-machine learning models from the plurality of data centers. Meanwhile, sensitive information may include personal information. Thus, in the following description, the terms “sensitive information” and “personal information” may be used together.

The machine learning apparatus 200 may perform step 320 of obtaining an integrated machine learning model based on the plurality of sub-machine learning models. For example, the machine learning apparatus 200 may obtain an integrated machine learning model by combining the plurality of sub-machine learning models.

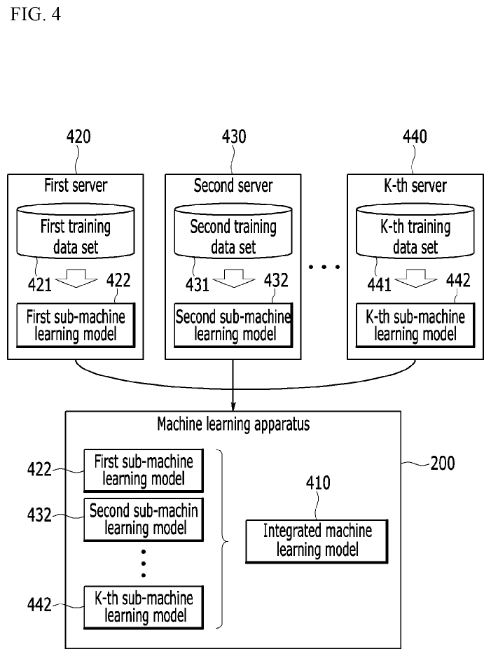

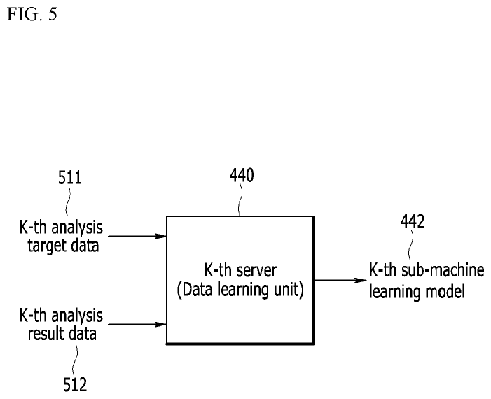

Hereinafter, more detailed description on steps 310 and 320 will be followed with reference to FIG. 4 and FIG. 5.

FIG. 4 is a diagram showing a process of obtaining an integrated machine learning model according to an embodiment of the present disclosure.

A first data center to a k-th data center may be different medical institutions. In FIG. 4, a first server 420 to a k-th server 440 may be servers included in a first data center to a k-th data center, respectively. The first server 420 to the k-th server 440 may be located in different positions. For example, the first server 420 to the k-th server 400 may be in different positions physically, functionally, or structurally. In addition, each of the first server 420 to the k-th server may not directly share data with any other of the first server 420 to the k-th server. The first server 420 to the k-th server 440 may store or process the data obtained by the first data center to the k-th data center, respectively. Each of the first server 420 to the k-th server 440 may not share data with each other.

Otherwise, the first server 420 to the k-th server 440 may share only some of the data. For example, the first server 420 to the k-th server 440 may not share sensitive data including personal information. However, the first server 420 to the k-th server 440 may share insensitive data.

A plurality of training data sets may be obtained independently from each other. That is, a first training data set 421, a second training data set 431, and the k-th training data set 441 included in the plurality of training data sets may be data obtained in the first server 420, the second server 430, and the k-th server 440, respectively.

The plurality of training data sets may contain sensitive information, which may make it difficult to be shared. The first training data set 421 may be processed only in the first server 420. The second training data set 431 may be processed only in the second server 430. The k-th training data set 441 may be processed only in the k-th server 440. Accordingly, the servers may perform processes to transform the plurality of training data sets to be sharable.

The plurality of training data sets may be independent from each other. The first training data set 421 obtained in the first server 420 may not affect the second training data set 431 obtained in the second server 430. Similarly, the second training data set 431 obtained in the second server 430 may not affect the first training data set 421 obtained in the first server 420. First analysis target data and a first analysis result data included in the first training data set 421 may be independent of second analysis target data and a second analysis result data included in the second training data set 431, respectively.

This is because a data center including a plurality of servers acquires data in an independent manner Namely, a plurality of training data sets may be data related to medical images acquired in different environments.

For example, the first server to the k-th server may be located in the first data center to the k-th data center, respectively. A plurality of data centers may be located in different places. Data acquisition equipment in each of the plurality of data centers may be different or the same. The plurality of data centers may acquire data at independent times form each other. The data centers may acquire data from objects independent of each other. The data centers may obtain analysis results from independent medical professionals.

In addition, a plurality of data centers may generate data using an independent data acquisition set value or a data output set value even when the same equipment is used. When the data is a medical image, a medical imaging apparatus may obtain the medical image with various set values. In addition, a medical image display apparatus may output the medical image with various set values. The data may vary depending on the data acquisition set value and the data output set value.

However, a plurality of training data sets 421, 431, and 441 may be compatible with each other. As described above, the training data set may include an analysis target data and an analysis result data. The plurality of training data sets 421, 431, and 441 may include the same type of analysis target data. For example, the analysis target data may be CT, MRI, X-ray, mammography, or ultrasonic wave images. Further, the plurality of training data sets 421, 431, and 441 may include the analysis result data of the same type. For instance, the analysis result data may include information related to a lesion diagnosed based on the analysis target data by a medical professional.

When there is no compatibility among the plurality of training data sets, a plurality of servers 420, 430, and 440 may perform preprocessing of standardizing the plurality of training data sets to have compatibility thereamong.

The k-th server 440 may generate a k-th sub-machine learning model 442 by performing machine learning on the k-th training data set 441. For example, the first server 420 may generate a first sub-machine learning model 422 by performing machine learning on the first training data set 421. The second server 430 may generate a second sub-machine learning model 432 by performing machine learning on the second training data set 431. Hereinafter, more detailed description will be followed with reference to FIG. 5.

FIG. 5 is a diagram for explaining a process to generate a sub-machine learning model according to an embodiment of the present disclosure.

As described above, the k-th training data set 441 may include the k-th analysis target data 511 and the k-th analysis result data 512. For example, the k-th analysis target data 511 may include at least one of medical images of a patient and various measurement data related to a patient's condition. The k-th analysis target data 511 may include sensitive information.

The k-th analysis result data 512 may be a result diagnosed based on the k-th analysis target data 511 by a medical professional. The k-th analysis result data 512 may be ground-truth data or a ground-truth label. The k-th analysis result data 512 may correspond to the k-th analysis target data 511.

The k-th server 440 may include a data learning unit 110. The k-th server 440 may generate a k-th sub-machine learning model 442 by performing machine learning on a relationship between the k-th analysis target data 511 and the k-th analysis result data 512. The k-th server 440 may perform machine learning based on a deep neural network. The k-th server 440 may improve the accuracy of the k-th sub-machine learning model 520 while performing forward propagation and back propagation using the k-th analysis target data 511 and the k-th analysis result data 512.

In the above description, a generation process of the k-th sub-machine learning model 442 has been described focusing on the k-th server. A process that the first server 420 generates the first sub-machine learning model 422 and a process that the second server 430 generates the second sub-machine learning model 432 are similar to the process that the k-th server 430 generates the k-th sub-machine learning model 432. Therefore, repetitive explanation will be omitted in the following description.

The k-th sub-machine learning model 442 may be a machine learning model that has learned a relationship between the analysis target data and the analysis result data. Accordingly, the k-th sub-machine learning model 442 that completed machine learning may receive an input of new analysis target data and may output prediction result data. Specifically, the k-th server 440 may include a data recognition unit 120. The data recognition unit 120 of the k-th server 440 may acquire the new analysis target data not included in the k-th training data set and apply it to the k-th sub-machine learning model 442. The k-th sub-machine learning model 442 may output prediction result data corresponding to the new analysis target data. Here, applying the analysis target data to the sub-machine learning model represents that forward propagation of the sub-machine learning model is performed by using the analysis target data as an input. The prediction result data is a prediction result that the k-th sub-machine learning model 442 made and may be similar to the ground-truth data. The prediction result data is referred to as a “prediction label” and is a prediction result made by the machine learning model. There is a difference in that the ground-truth label or the ground-truth data are not information predicted by the machine learning model but information that the user derived through actual analysis.

The k-th sub-machine learning model 442 may be a machine learning model optimized for the k-th training data set. The k-th server 440 may analyze the analysis target data acquired by the k-th server using the k-th sub-machine learning model 442 and output the prediction result data with high accuracy. However, the k-th sub-machine learning model 442 may output prediction result data with relatively low accuracy for the analysis target data acquired by a server other than the k-th server 440. Similarly, the first sub-machine learning model 422 may be a machine learning model specialized for the first training data set. As a result, the first sub-machine learning model 422 may output prediction data with relatively low accuracy regarding the analysis target data obtained by the second server 430 or the k-th server 440.

Referring back to FIG. 4, the first server 420 to the k-th server 440 may transmit the generated the first sub-machine learning model 422 to the k-th sub-machine learning model 442, to the machine learning apparatus 200. The first sub-machine learning model 422 to the k-th sub-machine learning model 442 do not include sensitive information, for example, personal information, included in the first training data set 421 to the k-th training data set 44, and thus may be shared without any restraint. Transmission may be made using a wired or wireless communication device. In addition, the first sub-machine learning model 422 to the k-th sub-machine learning model 442 may be stored in a recording medium and transferred to the machine learning apparatus 200.

The machine learning apparatus 200 may perform step 310 of receiving a plurality of sub-machine learning models. The plurality of sub-machine learning models may include the first sub-machine learning model 422 to the k-th sub-machine learning model 442.

Specifically, in order to receive the plurality of sub-machine learning models, the machine learning apparatus 200 may perform a step of receiving the first sub-machine learning model that has performed machine learning based on the first training data set included in the plurality of training data sets. Also, the machine learning apparatus 200 may perform a step of receiving the second sub-machine learning model that has performed machine learning based on the second training data set included in the plurality of training data sets. The first sub-machine learning model 422 and the second sub-machine learning model 432 may be independently generated in the first server 420 and the second server 430, respectively.

Also, the machine learning apparatus 200 may perform step 320 of obtaining an integrated machine learning model 410 based on the plurality of sub-machine learning models. The machine learning apparatus 200 may generate an integrated machine learning model 410 by combining from the first sub-machine learning model 422 to the k-th sub-machine learning model 442. That is, the integrated machine learning model 410 may be a group of k machine learning models. For example, if the machine learning apparatus 200 receives the first sub-machine learning model 422 and the second sub-machine learning model 432, the machine learning apparatus 200 performs a step of obtaining the integrated machine learning model 410 by combining the first sub-machine learning model 422 with the second sub-machine learning model 432. In this case, since the machine learning apparatus 200 generates the integrated machine learning model 410 by using the training data sets from all data centers, the accuracy of the final machine learning model generated based on the integrated machine learning model 410 is also improved. That is, the final machine learning model may generate prediction result data by accurately analyzing a new analysis target data.

The machine learning apparatus 200 may determine one sub-machine learning model among the first sub-machine learning model 422 to the k-th sub machine learning model 442, as the integrated machine learning model 410. For example, when one data center among the plurality of data centers maintains the most generic data, the user or the machine learning apparatus 200 may determine a sub-machine learning model of the one data center as the integrated machine learning model 410. This is because the one data center may represent the plurality of data centers.

In addition, a sub-machine learning model of an arbitrary data center may be determined as the integrated machine learning model 410. For example, the first server 420 may obtain a first sub-machine learning model 422 by performing machine learning on a first training data set 421 including a first analysis target data and first analysis result data. The machine learning apparatus 200 may determine the first sub-machine learning model 422 as the integrated machine learning model 410. The integrated machine learning model 410 is a configuration for obtaining intermediate data such as feature data. Therefore, by using an arbitrary sub-machine learning model, the machine learning apparatus 200 may obtain the final machine learning model of the present disclosure.

Without passing through the processes shown in FIG. 3, the machine learning apparatus 200 may receive a sub-machine learning model generated in a server as described above. In this case, the machine learning apparatus 200 may reduce the amount of data to be transmitted via wired/wireless communication and save processing power of the server.

In addition, the machine learning apparatus 200 may generate an integrated machine learning model 410 by using at least one of the first sub-machine learning model 422 to the k-th sub-machine learning model 442. The machine learning apparatus 200 may determine a similarity among the first sub-machine learning model 422 to the k-th sub-machine learning model 442. The machine learning apparatus 200 may measure a similarity by randomly selecting two sub-machine learning models among the first sub-machine learning model 422 to the k-th sub-machine learning model 422. When the similarity is larger than or equal to a predetermined threshold, the machine learning apparatus 200 may generate an integrated machine learning model 410 by removing one of the two sub-machine learning models. For example, if two data centers are in very similar environments, the sub-machine learning models derived by the servers included in each data center may be similar to each other. The machine learning apparatus 200 may generate the integrated machine learning model 410 by performing the above-described process for all combinations of the two sub-machine learning models selected among the first sub-machine learning model 422 to the k-th sub-machine learning model 442. As a result, the machine learning apparatus 200 can reduce the data throughput of the processor and the burden of data transmission and reception, by removing the redundant sub-machine learning models.

In addition, the machine learning apparatus 200 may generate the integrated machine learning model 410, without using at least one of the first sub-machine learning model 422 to the k-th sub-machine learning model 442. The machine learning apparatus 200 may generate an arbitrary basic model as the integrated machine learning model 410. In this case, since the machine learning apparatus 200 may not perform the processes as shown in FIG. 3, the throughput of the processor may be reduced and the burden of data transmission may be decreased.

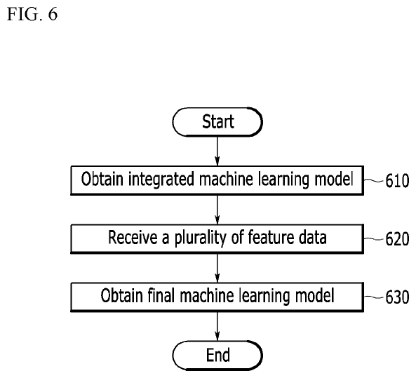

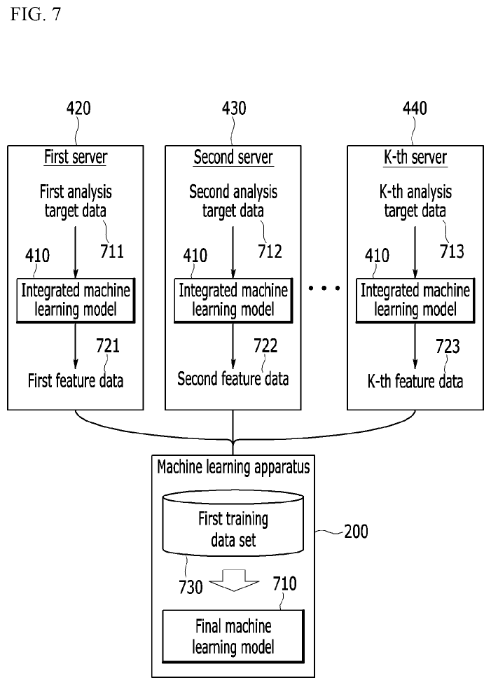

FIG. 6 is a flowchart showing a method for generating a final machine learning model according to an embodiment of the present disclosure. FIG. 7 is a diagram for explaining a method for generating a final machine learning model according to an embodiment of the present disclosure.

Referring to FIG. 6, the machine learning apparatus 200 may perform step 610 of obtaining an integrated machine learning model 410. Hereinafter, since the step of obtaining the integrated machine learning model 410 has been described above, repetitive description will be omitted hereinafter.

The machine learning apparatus 200 may transmit the integrated machine learning model 410 to each of the first server 420 to the k-th server 440. The first server 420 to the k-th server 440 may encrypt a plurality of analysis target data 711, 712, and 713 by using the integrated machine learning model 410. Sensitive information such as personal information may not be identified in a plurality of the encrypted analysis target data. In addition, the plurality of encrypted analysis target data may not be completely reconstructed to the plurality of analysis target data 711, 712, and 713. A first feature data 721 to the k-th feature data 723 may be data obtained through lossy compression of the first analysis target data 711 to the k-th analysis target data 713, respectively. Since the first feature data 721 to the k-th feature data 723 are lossy-compressed data, the sensitive information, for example, personal information may not be identified therein. In addition, the first feature data 721 to the k-th feature data 723 may not be completely reconstructed as the first analysis target data 711 to the k-th analysis target data 713, respectively. The plurality of analysis target data 711, 712, and 713 may not be sharable, because personal information is included therein. However, the first feature data 721 to the k-th feature data 723 may be sharable because personal information is not identified therein. In addition, since each of the first feature data 721 to the k-th feature data 723 includes smaller amount of data than each of the first analysis target data 711 to the k-th analysis target data 713, the amount of data to be processed by the processor may be reduced.

The first server 420 may obtain the first feature data 721 by applying the first analysis target data 711 to the integrated machine learning model 410. The second server 430 may obtain the second feature data 722 by applying the second analysis target data 712 to the integrated machine learning model 410. The k-th server 440 may obtain the k-th feature data 723 by applying the k-th analysis target data 713 to the integrated machine learning model 410. The first server 420 to the k-th server 440 may transmit the first feature data 721 to the k-th feature data 723, to the machine learning apparatus 200, respectively.

A plurality of analysis result data may correspond to the plurality of analysis target data 711, 712, and 713, respectively. However, when the plurality of analysis target data 711, 712, and 713 are encrypted, it is not specified to whom the plurality of the analysis result data belongs. Therefore, the first server 420 to the k-th server 440 may not additionally encrypt the analysis result data. The first server 420 to the k-th server 440 may transmit the analysis result data to the machine learning apparatus 200.

The first feature data 721 to the k-th feature data 723 may not be prediction result data. That is, each of the first feature data 721 to the k-th feature data 723 may not be the analysis result corresponding to the plurality of the analysis target data 711, 712, and 713, which the first server 420 to the k-th server 440 derives using the integrated machine learning model 410, respectively. However, the present disclosure is not limited thereto, and the first feature data 721 to the k-th feature data 723 may be analysis result corresponding to the plurality of analysis target data 711, 712 and 713, which the first server 420 to the k-th server 440 derives using the integrated machine learning model 410

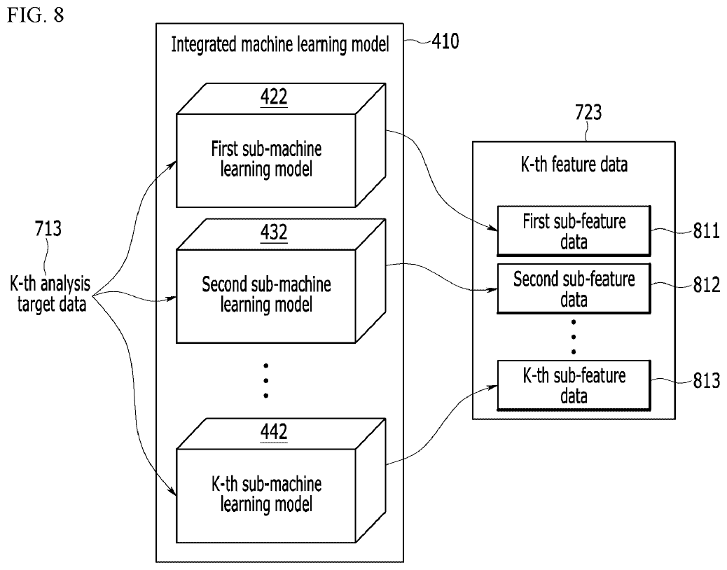

Hereinafter, detailed description of the first feature data 721 to the k-th feature data 723 is followed with reference to FIG. 8 to FIG. 10.

FIG. 8 is a diagram for explaining feature data according to an embodiment of the present disclosure.

FIG. 8 is a diagram showing a data processing process performed in the k-th server 440 shown in FIG. 7.

Referring to FIG. 8, an integrated machine learning model 410 may include a first sub-machine learning model 422 to a k-th sub-machine learning model 442. In FIG. 8, the integrated machine learning model 410 is represented as including the first sub-machine learning model 422 to the k-th sub-machine learning model 442, but should not be construed to be limited thereto. As described above, the machine learning apparatus 200 may generate an integrated machine learning model 410 by using at least one of the first sub-machine learning model 422 to the k-th sub-machine learning model 442. Moreover, the integrated machine learning model 410 may be a predetermined basic model.

The k-th server 440 may apply the k-th analysis target data 713 to the integrated machine learning model 410. The k-th server 440 may obtain a first sub-feature data 811 to a k-th sub-feature data 813, by applying the k-th analysis target data 713 to the first sub-machine learning model 422 to the k-th sub-machine learning model 442.

The k-th server 440 may acquire the first sub-feature data 811 by applying the k-th analysis target data 713 to the first sub-machine learning model 422. The k-th server 440 may acquire the second sub-feature data 812 by applying the k-th analysis target data 713 to the second sub-machine learning model 432. The k-th server 440 may acquire the k-th sub-feature data 813 by applying the k-th analysis target data 713 to the k-th sub-machine learning model 442. The k-th feature data 723 may include the first sub-feature data 811 to the k-th sub-feature data 813. Since the k-th analysis target data 713 includes personal information and the k-th feature data 723 is encrypted, personal information may not be identified. Accordingly, the k-th feature data 723 may be transmitted to the outside.

The first server 420 may perform the same process as the k-th server 440 and acquire the first feature data 721 from the first analysis target data 711. The first server 420 may acquire a first first sub-feature data by applying the first analysis target data 711 to the first sub-machine learning model 422. The first server 420 may obtain first second sub-feature data by applying the first analysis target data 711 to the second sub-machine learning model 432. The first server 420 may obtain first k-th sub-feature data by applying the first analysis target data 711 to the k-th sub-machine learning model 442. The first feature data 721 may include the first first sub feature data, the first second sub feature data, and the first k-th sub feature data.

Additionally, the second server 430 may perform the same process as the k-th server 440 and acquire the second feature data 722 from the second analysis target data 712. The second server 430 may acquire second first sub-feature data by applying the second analysis target data 712 to the first sub-machine learning model 422. The second server 430 may acquire second second sub-feature data by applying the second analysis target data 712 to the second sub-machine learning model 432. The second server 430 may acquire second k-th sub-feature data by applying the second analysis target data 712 to the k-th sub-machine learning model 442. The second feature data 722 may include the second first sub-feature data, the second second sub-feature data, and the second k-th sub-feature data.

The first analysis target data 711 and the second analysis target data 712 may include personal information. In addition, since the first feature data 721 and the second feature data 722 are encrypted, personal information may not be identified. As a result, the first feature data 721 and the second feature data 722 may be transmitted to the outside. The first server 420 to the k-th server 440 may transmit the first feature data 721 to the k-th feature data 723, to the machine learning apparatus 200.

In the above description, the integrated machine learning model 410 is described as including the first sub-machine learning model 422 to the k-th sub-machine learning model 442, but the present disclosure should not be construed as to be limited thereto. The integrated machine learning model 410 may be generated by using at least one of the first sub-machine learning model 422 to the k-th sub-machine learning model 442. In addition, the integrated machine learning model 410 may be a predetermined basic model.

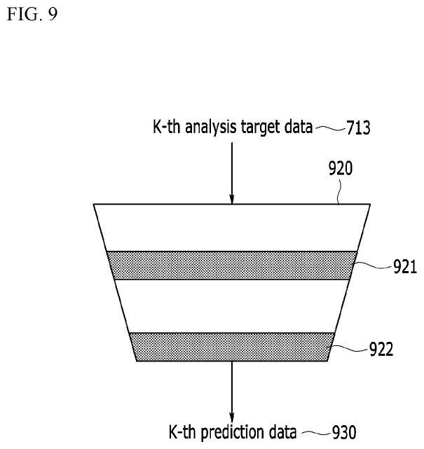

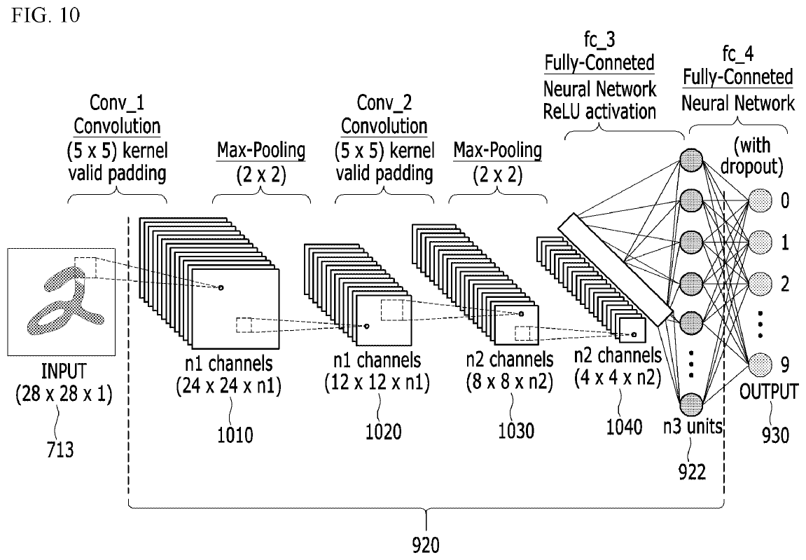

Hereinafter, a process for generating encrypted feature data is described in more detail with reference to FIG. 9 and FIG. 10.

FIG. 9 is a diagram for explaining a process of generating feature data according to an embodiment of the present disclosure.

FIG. 9 is a diagram illustrating a process through which the k-th server shown in FIG. 8 generates k-th sub-feature data 813 by applying k-th analysis target data 713 to k-th sub-machine learning model 442.

As described above, applying the k-th analysis target data 713 to the integrated machine learning model refers to performing forward propagation by inputting the k-th analysis target data 713 to a sub-machine learning model included in an integrated machine learning model.

When the k-th server 440 performs forward propagation of the k-th target analysis data 713 to the k-th sub-machine learning model 442, feature data 920 including a plurality of layers may be generated. Each layer of the feature data 920 including a plurality of layers may be generated by performing lossy compression of the k-th analysis target data 713. In addition, feature data 920 including a plurality of layers may be data obtained through transforming the k-th analysis target data 713 so as not to be recognized by a user. That is, the feature data 920 including a plurality of layers may be data obtained by encrypting the k-th analysis target data 713. A layer included in the feature data 920 may not be completely reconstructed to the k-th analysis target data 713.

Also, when the k-th server 440 performs forward propagation of the k-th analysis target data 713 to the k-th sub-machine learning model 442, k-th prediction data 930 may be generated finally. The k-th prediction data 930 may be prediction information obtained through analyzing the k-th analysis target data. The k-th sub-feature data 813 shown in FIG. 8 may be different from the k-th prediction data 930, but it is not limited thereto. Otherwise, the k-th sub-feature data 813 may be the same as the k-th prediction data 930.

The k-th server 440 may select feature data of at least one layer among the plurality of layers constituting the feature data 920. According to a predetermined method, the k-th server 440 may determine an intermediate layer 921 or a final layer 922 of the plurality of layers as the k-th sub-feature data 813.

The k-th server 440 may perform the same process as above-described, for all of the first sub-machine learning model 422 to the k-th sub-machine learning model 442 included in the integrated machine learning model 410.

In addition, the k-th server 440 may acquire the first sub-feature data 811 to the k-th sub-feature data 813 by selecting the feature data at the same positions among the feature data including a plurality of layers, which are generated by applying the first sub-machine learning model 422 to the k-th sub-machine learning model 442 to the k-th analysis target data 713.

However, the present disclosure is not limited thereto, and the k-th server 440 may select feature data at positions determined in different manners, in the feature feature data 920 including a plurality of layers that are generated by applying the first sub-machine learning model 422 to the k-th sub-machine learning model 442 to the k-th analysis target data 713. According to a predetermined method, the k-th server 440 may determine a position of a layer to be selected from the plurality of layers obtained based on the sub-machine learning model.

In the above description, the process is explained to be performed by the k-th server 440, but the same process may be performed by the first server 420 and the second server 430. Thus, repetitive description will be omitted.

Hereinafter, more detailed description will be followed with reference to FIG. 10 that shows FIG. 9 more specifically.

FIG. 10 is a diagram for explaining a feature data generation process according to an embodiment of the present disclosure.

In FIG. 10, “INPUT” may refer to the k-th analysis target data 713. The k-th server 440 may apply the k-th sub-machine learning model 442 to the k-th analysis target data 713 through the following process.

The k-th server 440 may obtain a first layer 1010 with the size of 24×24×n1 by performing a first convolution Conv_1 on the k-th analysis target data 713. The k-th server 440 may obtain a second layer 1020 with the size of 12×12×n1 by performing max pooling on the first layer 1010. The k-th server 440 may obtain a third layer 1030 with the size of 8×8×n2 by performing a second convolution Conv_2 on the second layer 1020. The k-th server 440 may obtain a fourth layer 1040 with the size of 4×4×n2 by performing max pooling on the third layer 1030. The k-th server 440 may obtain a final layer 922 with the size of n3 by applying a first fully connected neural network fc_3 to the fourth layer 1040. Also, the k-th server 440 may transmit “OUTPUT” 930 by applying a second fully connected neural network fc_4 to the final layer 922. The OUTPUT 930 may be a prediction label corresponding to an INPUT predicted based on the k-th sub-machine learning model 442 or a predicted analysis result.

Here, the first layer 1010 to the fourth layer 1040 and the final layer 922 may be included in generated the plurality of layers, if the k-th analysis target data 713 is forward-propagated to the k-th sub-machine learning model 442. Based on a predetermined method, the k-th server 440 may obtain the k-th sub-feature data 813 by selecting at least one layer among a plurality of feature layers 1010, 1020, 1030, 1040, and 922. For example, the k-th server 440 may obtain the k-th sub-feature data 813 by selecting at least one layer at a predetermined position. Here, the position refers to a position of a layer with respect to the plurality of layer 1010, 1020, 1030, 1040, and 922.

A first convolution, a second convolution, a max pooling, a first fully connected neural network fc_3, and a second fully connected neural network fc_4 may be components included in the k-th sub-machine learning model 442. FIG. 10 shows an embodiment of the present disclosure. Here, a k-th machine learning model may further include various components, and may be a model where some components are deleted from the embodiment shown in FIG. 10. In addition, an optimized k-th machine learning model having a plurality of updated weights related to the first convolution, the second convolution, the first fully connected neural network fc_3, and the second fully connected neural network fc_4 may be obtained through forward propagation and back propagation during a machine learning process of the k-th machine learning model.

For generalization, a case where the k-th server performs forward propagation of the k-th analysis target data 713 to the k-th sub-machine learning model 442 is described with reference to FIG. 9 and FIG. 10. However, in a case where the k-th server performs forward propagation of the k-th analysis target data 713 to the k-th sub-machine learning model 442 to the k-th sub-machine learning model 442, a plurality of layers may be generated in the same way. Also, the k-th server 440 may acquire the first sub-feature data 811 to the k-th sub-feature data 813 by selecting at least one layer from the plurality of layers based on a predetermined method. The k-th server 440 may acquire the first sub-feature data 811 to the k-th sub-feature data 813 by selecting a layer with the same position among the plurality of layers. For example, the k-th server 440 may acquire the first sub-feature data 811 to the k-th sub-feature data 813 by selecting a final layer 922 among the plurality of layers. The k-th server 440 may acquire the k-th feature data 723 based on at least one of the first sub-feature data 811 to the k-th sub-feature data 813.

Also, the first server 420 and the second server 430 may operate in the same way as the k-th server 440. For example, even when the first server 420 performs forward propagation of the first analysis target data 711 to the first sub-machine learning model 422 to the k-th sub-machine learning model 442, a plurality of layers may be generated in the same way. The first server 420 may acquire the first first sub-feature data to the first k-th sub-feature data by selecting at least one layer based on a predetermined method. In addition, the first server 420 may acquire the first feature data 721 based on at least one of the first first sub-feature data to the first k-th sub-feature data.

Even when the second server 430 performs forward propagation of the second analysis target data 712 to the first sub-machine learning model 422 to the k-th sub-machine learning model 442, a plurality of layers may be generated in the same manner. The second server 430 may acquire second first sub-feature data to second k-th sub-feature data, by selecting at least one layer based on a predetermined method. Moreover, the second server 430 may acquire the second feature data 722 based on at least one of the second first sub-feature data to second k-th sub-feature data. The first server 420 to the k-th server 440 may transmit the generated first feature data 721 to the k-th feature data 723 to the machine learning apparatus 200.

Hereinafter, more detailed description on a process of selecting at least one layer based on a predetermined method will be followed. Also, for convenience of explanation, a case where only the first server 420 and the second server 430 are provided will be described.

Referring to FIG. 7, the first feature data 721 may be acquired using at least one sub-machine learning model included in the integrated machine learning model 410. The first feature data 721 may include at least one feature layer among a plurality of first feature layers obtained by applying the first analysis target data 711 to the first sub-machine learning model. At least one feature layer selected from the plurality of first feature layers may be first first sub-feature data. Further, the first feature data 721 may include at least one feature layer from a plurality of second feature layers obtained by applying the first analysis target data to the second sub-machine learning model. At least one feature layer selected from the plurality of second feature layers may be the first second sub-feature data. The first feature data 721 may be acquired by combining the first first sub-feature data and the first second sub-feature data. For example, when the first first sub-feature data includes “a” feature layers and the first second sub-feature data includes “b” feature layers, the first feature data 721 may include “a+b” feature layers.

In addition, the first feature data 721 may be data obtained by performing an operation on the first first sub-feature data and the first second sub-feature data. The operation may include bitwise operators such as AND, OR, or XOR, or arithmetic operations including addition, subtraction, multiplication, and division. The server may acquire the first feature data 721 by performing an operation between elements included in first first sub-feature data and the first second sub-feature data. The same process is performed to acquire the second feature data 722 to k-th feature data 723.

Also, referring to FIG. 7, the second feature data 722 may be acquired using at least one sub-machine learning model in the integrated machine learning model 410. The second feature data 722 may include at least one feature layer among a plurality of third feature layers obtained by applying the second analysis target data 712 to the first sub-machine learning model 422. At least one feature layer among the plurality of third feature layers may be the second first sub-feature data. In addition, the second feature data 722 may include at least one feature layer of a plurality of fourth feature layers obtained by applying the second analysis target data 712 to the second sub-machine learning model 432. At least one of the plurality of fourth feature layers may be the second second sub-feature data. The second feature data 722 may be obtained by combining the second first sub-feature data and the second second sub-feature data.

At this time, in order to obtain the first feature data and the second feature data, the machine learning apparatus 200 or the server may select a layer at the same position in the plurality of first feature layers, the plurality of second feature layers, the plurality of third feature layers and the plurality of fourth feature layers. Referring to FIG. 9, the machine learning apparatus 200 or the server may acquire the first feature data and the second feature data using one of a mid-layer 921 or a final layer 922.

For example, a case where the first feature data and the second feature data are acquired using the final layer 922 will be described below. The plurality of first feature layers may include a plurality of feature layers 1010, 1020, 1030, 1040, and 922 shown in FIG. 10. The machine learning apparatus 200 or the first server 420 may acquire a first first sub-feature layer by selecting the final layer 922 from the plurality of first feature layers. In addition, the plurality of second feature layers may include a plurality of feature layers 1010, 1020, 1030, 1040, and 922 shown in FIG. 10. The machine learning apparatus 200 or the first server 420 may obtain a first second sub-feature layer by selecting the final layer 922 among the plurality of second feature layers. The first feature data may include a first first sub feature layer and a first-second sub feature layer. The plurality of third feature layers may include a plurality of feature layers 1010, 1020, 1030, 1040, and 922 shown in FIG. 10. The machine learning apparatus 200 or the second server 430 may acquire a second first sub-feature layer by selecting the final layer 922 from the plurality of third feature layers. Also, the plurality of fourth feature layers may include the plurality of feature layers of 1010, 1020, 1030, 1040, and 922 shown in FIG. 10. The machine learning apparatus 200 or the second server 430 may acquire a second second sub-feature layer by selecting the final layer 922 among the plurality of fourth feature layers. The second feature data may include the second first sub-feature layer and the second second sub-feature layer.

Hereinafter, the amount of the data received by the machine learning apparatus 200 will be described. It is assumed that each server includes “a” analysis target data and the number of servers is “b”. Since the number of sub-machine learning models included in the integrated machine learning model 410 is the same as the number of servers, it may be “b”. When “b” sub-machine learning models are applied to “a” analysis target data of one server, a×b″ sub-feature data may be generated in one server. Also, since the same number of sub-feature data are generated for the “b” servers, a total number of “a×b×b” sub-feature data may be generated. In the above description, it is assumed that the number of sub-machine learning model is the same as that of servers, but it is not limited thereto. One or more sub-machine learning model may be included in the integrated machine learning model 410. This is because the integrated machine learning model 410 may be one basic model or at least one sub-machine learning model selected from a plurality of sub-machine learning models. In this case, the sub-feature data may be less than or equal to “a×b×b”.

Referring back to FIG. 6 and FIG. 7, the first server 420 to the k-th server 440 may transmit the first feature data 721 to the k-th feature data 723, to the machine learning apparatus 200. In addition, the first server 420 to the k-th server 440 may transmit analysis result data to the machine learning apparatus 200. The machine learning apparatus 200 may perform a step 620 of receiving a plurality of feature data 721, 722, and 723 acquired by applying the integrated machine learning model 410 to the plurality of analysis target data 711, 712, and 713. Here, the plurality of analysis target data 711, 712, and 713 are included in a plurality of training data sets, each of which is independent from each other.

For example, the machine learning apparatus 200 may perform a step of receiving the k-th feature data 723 obtained by applying the integrated machine learning model 410 to the k-th analysis target data 713. In the k-th feature data 723, personal information identifiable in the k-th analysis target data 713 may be encrypted. Therefore, the personal information may not be identified in the k-th feature data 723. Similarly, the machine learning apparatus 200 may perform a step of receiving first feature data acquired by applying an integrated machine learning model to first analysis target data. Also, the machine learning apparatus 200 may perform a step of receiving second feature data obtained by applying the integrated machine learning model to second analysis target data.

As described above, the training data set may include the analysis target data and the analysis result data. Meanwhile, the analysis target data cannot be taken out from the data center. Thus, the first server to the k-th server may transmit a plurality of feature data 721, 722 and 723 to the machine learning apparatus 200, instead of the plurality of analysis target data 711, 712 and 713. This is because the personal information is not identified from the plurality of the feature data 721, 722, and 723, and the plurality of the feature data 721, 722, and 723 cannot be reconstructed to the analysis target data. In addition, the machine learning apparatus 200 may receive a plurality of analysis result data corresponding to each of the plurality of feature data 721, 722, and 723, from each of the first server to the k-th server, respectively. The plurality of analysis result data may not be encrypted. The plurality of analysis result data may be used as ground-truth data or a ground-truth label. In addition, the plurality of analysis result data corresponds to the plurality of feature data 721, 722, and 723, respectively, and the personal information is not identified from the plurality of feature data 721, 722, and 723. Thus, it may not be specified to whom the plurality of the analysis result data belongs. The machine learning apparatus 200 may acquire the plurality of feature data 721, 722, and 723 and the plurality of analysis result data, as a final data set 730.

The machine learning apparatus 200 may perform step 630 of obtaining a final machine learning model by performing machine learning on a relationship between the plurality of feature data 721, 722, and 723, and a plurality of analysis result data included in the plurality of training data set.

More specifically, the machine learning apparatus 200 may perform a step of obtaining the final machine learning model 710 by performing machine learning on a correlation between the first feature data 721 and the first analysis result data, a correlation between the second feature data 722 and the second analysis result data, or a correlation between the k-th feature data 723 and the k-th analysis result data.

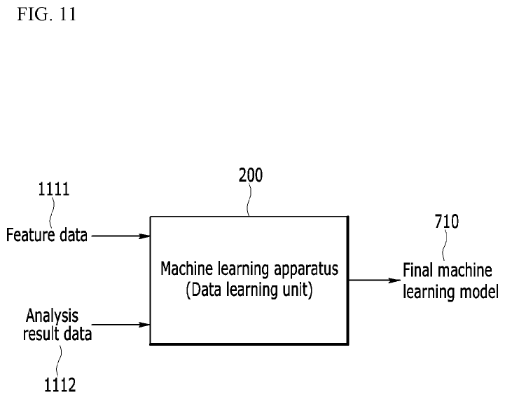

FIG. 11 is a diagram showing a process of generating a final machine learning model 710 according to an embodiment of the present disclosure.

As described above, a final data set 730 may include a feature data 1111 and an analysis result data 1112. The feature data 1111 may include the first feature data 721 to the k-th feature data 723 shown in FIG. 7. Each of the first feature data 721 to the k-th feature data 723 may have one-to-one correspondence with each of the first analysis target data 711 to the k-th analysis target data 713. The analysis result data 1112 may be ground-truth data or a ground-truth label. The analysis result data 1112 may include the first analysis result data to the k-th analysis result data. Each of the first analysis result data to the k-th analysis result data may have a one-to-one correspondence with each of the first analysis target data to the k-th analysis target data. In addition, each of the first analysis result data to the k-th analysis result data may have a one-to-one correspondence with each of the first feature data 721 to the k-th feature data 723.

The machine learning apparatus 200 may include a data learning unit 110. The machine learning apparatus 200 may generate a final machine learning model 710 by performing machine learning on a relationship between the feature data 1111 and the analysis result data 1112. The machine learning apparatus 200 may perform machine learning based on a deep neural network. The machine learning apparatus 200 may improve the accuracy of the final machine learning model 710 while performing forward propagation and back propagation using the feature data 1111 and the analysis result data 1112.

The final machine learning model 710 may be a machine learning model that learns the relationship between feature data 1111 and analysis result data 1112. Accordingly, the final machine learning model 710 having completed machine learning may receive a new feature data and output prediction result data.

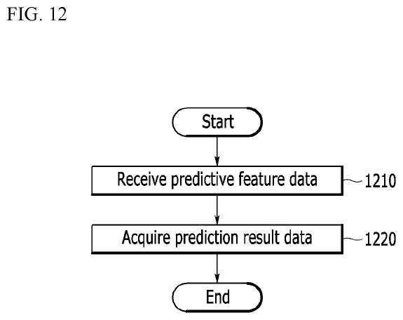

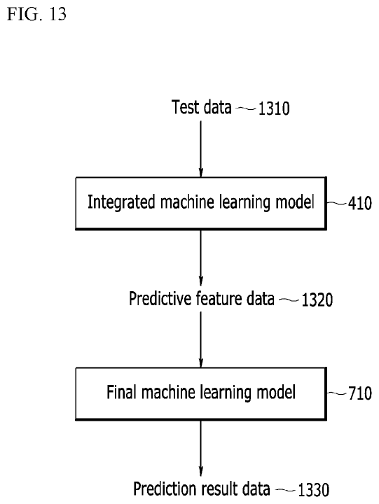

A process that the final machine learning model 710 analyzes new feature data will be described in more detail with reference to FIG. 12 and FIG. 13.

FIG. 12 is a flowchart showing an operation of a machine learning apparatus according to an embodiment of the present disclosure. FIG. 13 is a diagram illustrating an operation of a machine learning apparatus according to an embodiment of the present disclosure.

Specifically, the machine learning apparatus 200 may include a data recognition unit 120. A server may perform a step of acquiring predictive feature data 1320 by applying test data 1310 to an integrated machine learning model. Here, the server may be any of the first server 420 to the k-th server 440. Test data 1310 may be analysis target data newly acquired by the server. The server may not hold any analysis result data corresponding to the newly acquired analysis target data. The test data 1310 may include personal information.

The server may obtain predictive feature data 1320 by applying test data 1310 to the integrated machine learning model 410. The integrated machine learning model 410 may be the same as the integrated machine learning model 410 used for generating the final machine learning model 710.

The predictive feature data 1320 may be obtained by lossy compression of the test data 1310. The predictive feature data 1320 may be at least one layer among a plurality of layers generated by applying the test data 1310 to the integrated machine learning model. The predictive feature data 1320 may be generated through a process as shown in FIG. 8 to FIG. 10. For example, test data 1310 and the predictive feature data 1320 may correspond to the k-th analysis target data 713 and the k-th feature data 723 shown in FIG. 8, respectively. The predictive feature data 1320 may have been encrypted. Therefore, personal information may not be identified in the predictive feature data 1320. Since the personal information is not identified from the predictive feature data 1320, the server may take out the predictive feature data without restraints. The server may transmit the predictive feature data 1320 to the machine learning apparatus 200. The taken out predictive feature data 1320 may be rapidly and accurately processed by using an external high performance apparatus. The external high performance apparatus may be a machine learning apparatus 200.

The machine learning apparatus 200 may perform step 1210 of receiving the predictive feature data 1320 acquired by applying test data 1310 to an integrated machine learning model. Further, the machine learning apparatus 200 may perform a step 1220 of acquiring predictive result data 1330 related to test data 1310 by applying the predictive feature data 1320 to the final machine learning model 710. The predictive result data 1330 may be an analysis result of the test data 1310 by the final machine learning model 710. The predictive result data 1330 may be an analysis result of the machine learning apparatus 200 for the test data 1310. The predictive result data 1330 may be data automatically derived by the machine learning apparatus 200 without any user assistance. Since the final machine learning model 710 derives the result data by using all the data of the first server 420 to the k-th server 440, the result data may be similar to the ground truth data with high probability. In addition, according to the present disclosure, since the final machine learning model 710 derives the result data by using all the data of the first server 420 to the k-th server 440, predictive result data 1330 with high accuracy may be acquired no matter which data of the first server to the k-th server is used. In addition, since a large amount of predictive feature data 1320 is processed using an external machine learning apparatus 200 having higher performance than the first server 420 to the k-th server 440, the predictive result data 1330 may be obtained rapidly and accurately.

The user can derive a final analysis result for the test data 1310 by referring to the predictive result data 1330 derived by the machine learning apparatus 200. Due to the help of the machine learning apparatus 200, a user may derive the final analysis result more accurately.

In the above description, a configuration where a machine learning apparatus 200 generates an integrated machine learning model 410 using at least one of the first sub-machine learning model 422 to the k-th sub-machine learning model 442 and generates a final machine learning model 710 by using the generated integrated machine learning model has been described. However, as already described, the integrated machine learning model 410 may include a predetermined basic model. The machine learning apparatus 200 may generate the final machine learning model 710 based on the predetermined basic model, without performing a process of generating the integrated machine learning model 410. That is, the integrated machine learning model 410 shown in FIG. 6 to FIG. 13 may be replaced by a basic model.

Hereinafter, a process of generating a final machine learning model based on a basic model included in an integrated machine learning model 410 will be briefly described with reference to FIG. 7.

Referring to FIG. 7, the machine learning apparatus 200 may perform a step of receiving first feature data 721 acquired by applying a basic model to first analysis target data 711. In addition, the machine learning apparatus 200 may perform a step of receiving second feature data 722 acquired by applying the basic model to second analysis target data 712.

Here, the first analysis target data 711 and the second analysis target data 712 may be data related to medical images obtained in different environments. A general machine learning model for image classification may be used as the basic model. The basic model may be a pre-learned model. As the basic model, for example, ImageNet may be used. First analysis result data may be a result obtained through analyzing the first analysis target data 711 by a user, and the second analysis result data may be a result obtained through analyzing the second analysis target data 711 by the user. The first analysis result data and the second analysis result data may be ground truth data or a ground truth label. The first analysis target data 711 and the first analysis result data may be included in a first training data set 421. The second analysis target data 712 and the second analysis result data may be included in a second training data set 431.

The first feature data 721 and the second feature data 722 may be obtained through lossy compression of the first analysis target data 711 and the second analysis target data 712. The first feature data 721 and the second feature data 722 may be encrypted data of the first analysis target data 711 and the second analysis target data 712. The first feature data 721 and the second feature data 722 may not be completely reconstructed to the first analysis target data 711 and the second analysis target data 712, respectively. The first analysis target data and the second analysis target data may include personal information, and the personal information may not be identified in the first feature data and the second feature data. Therefore, in order to protect the personal information, the servers 420, 430, and 440 cannot transmit the first analysis target data 711 and the second analysis target data 712 to the machine learning apparatus 200. However, the servers 420, 430, and 440 may transmit the first feature data and the second feature data to the machine learning apparatus 200.

The first feature data 721 may be at least one layer among a plurality of first feature layers obtained by applying the first analysis target data 711 to the basic model. Likewise, the second feature data 722 may be at least one layer among a plurality of second feature layers obtained by applying the second analysis target data 712 to the basic model. The machine learning apparatus 200 may acquire the first feature data 721 and the second feature data 722 by selecting a layer having the same position from the plurality of first feature layers and the plurality of second feature layers.

The machine learning apparatus 200 may receive the first feature data 721, the first analysis result data, the second feature data 722 and the second analysis result data from the servers 420, 430, and 440.

The machine learning apparatus 200 may perform a step of obtaining a final machine learning model 710 by performing machine learning on a correlation between the first feature data 721 and the first analysis result data, and a correlation between the second feature data 722 and the second analysis result data.

Referring to FIG. 13, the server may acquire predictive feature data 1320 by applying a basic model to test data 1310. The machine learning apparatus 200 may perform a step of receiving the predictive feature data 1320 acquired by applying the basic model to the test data 1310. The machine learning apparatus 200 may perform a step of obtaining predictive result data 1330 corresponding to the test data 1310 by applying the final machine learning model 710 to the predictive feature data 1320.

Here, the predictive feature data may be obtained by lossy compression of the test data. The test data may include personal information, and the personal information may not be identified in the predictive feature data.

The predictive result data 1330 is an analysis result of the machine learning apparatus 200 for test data 1310. The predictive result data 1330 may be data automatically derived by the machine learning apparatus 200 without user assistance. Since the final machine learning model 710 derives the predictive result data by using all the data in the first server 420 to the k-th server 440, the predictive result data may be similar to ground truth data with a high probability. In addition, according to the present disclosure, since the final machine learning model 710 derives the predictive result data by using all the data in the first server 420 to the k-th server 440, the predictive result data 1330 with high accuracy may be obtained no matter which data in the first server to the k-th server is used. In addition, since a large amount of predictive feature data 1320 is processed using an external machine learning apparatus 200 having higher performance than any of the first server 420 to the k-th server 440, the predictive result data 1330 may be obtained rapidly and accurately.

In the above description, the present disclosure has been described with reference to the various embodiments thereof. It will be understood by the person of ordinary skill in the art that various modifications may be made without departing from the spirit and scope of the present disclosure as defined by the following claims. Therefore, the embodiments should be considered in a descriptive sense only and not for the purposes of limitation. Although the embodiments of the present invention have been described in detail above, the scope of the present invention is represented in the claims. All the difference falling within an equivalent range should be interpreted to be included in the present invention.

Meanwhile, the above-described embodiment of the present invention can be written as a program executable in a computer, and may be implemented on a general purpose digital computer that operates the program using a computer-readable recording medium. The computer-readable recording medium may include a magnetic storage medium (e.g., ROM, floppy disk, hard disk, and the like), and an optical reading medium (e.g., CD-ROM, DVD, and the like).

What is claimed is:

1. A machine learning method comprising:
receiving first feature data obtained by applying a basic model to first analysis target data;
receiving second feature data obtained by applying the basic model to second analysis target data; and
obtaining a final machine learning model through performing machine learning on a correlation between the first feature data and first analysis result data and a correlation between the second feature data and second analysis result data.

2. The method of claim 1, further comprising:
receiving predictive feature data obtained by applying the basic model to test data; and
obtaining prediction result data corresponding to the test data by applying the final machine learning model to the predictive feature data.

3. The method of claim 1, wherein the first analysis target data and the second analysis target data are related to medical images obtained in different environments.

4. The method of claim 2, wherein the first feature data, the second feature data, and the predictive feature data are obtained by performing lossy compression of the first analysis target data, the second analysis target data, and the test data, respectively.

5. The method of claim 2, wherein the first analysis target data, the second analysis target data and the test data include personal information, and the personal information is not identified in the first feature data, the second feature data, and the predictive feature data.

6. The method of claim 1, wherein the first analysis result data is a result obtained through analyzing the first analysis target data by a user, and the second analysis result data is a result obtained through analyzing the second analysis target data by the user.

7. The method of claim 1, wherein the first feature data is at least one layer of a plurality of first feature layers obtained by applying the first analysis target data to the basic model, and
wherein the second feature data is at least one layer of a plurality of second feature layers obtained by applying the second analysis target data to the basic model.

8. The method of claim 7, wherein the first feature data and the second feature data are obtained by selecting a layer located in a same position in the plurality of first feature layers and the plurality of second feature layers, respectively.

9. The method of claim 1, wherein the basic model is a previously learned machine learning model for image classification.

10. The method of claim 1, wherein the basic model is a sub-machine learning model that is obtained through machine learning of a first training data set including the first analysis target data and the first analysis result data.

11. A machine learning apparatus comprising:
a processor; and
a memory,
wherein, based on instructions stored in the memory, the processor
receives first feature data obtained by applying a basic model to first analysis target data,
receives second feature data obtained by applying the basic model to second analysis target data, and
obtains a final machine learning model through machine learning of a correlation between the first feature data and first analysis result data and a correlation between the second feature data and second analysis result data.

12. The machine learning apparatus of claim 11, wherein, based on the instructions stored in the memory, the processor
receives predictive feature data obtained by applying a basic model to test data, and
obtains predictive result data corresponding to the test data by applying the final machine learning model to the predictive feature data.

13. The machine learning apparatus of claim 11, wherein the first analysis target data and the second analysis target data are related to medical images obtained in different environments.

14. The machine learning apparatus of claim 12, wherein the first feature data, the second feature data, and the predictive feature data are obtained by lossy compression of the first analysis target data, the second analysis target data, and the test data, respectively.

15. The machine learning apparatus of claim 12, wherein the first analysis target data, the second analysis target data, and the test data include personal information, and the personal information is not identified in the first feature data, the second feature data, and the predictive feature data.

16. The machine learning apparatus of claim 11 wherein the first analysis result data is a result obtained through analyzing the first analysis target data by a user, and the second analysis result data is a result obtained through analyzing the second analysis target data by the user.

17. The machine learning apparatus of claim 11, wherein the first feature data is at least one layer of a plurality of first feature layers obtained by applying the first analysis target data to the basic model, and
wherein the second feature data is at least one layer of a plurality of second feature layers obtained by applying the second analysis target data to the basic model.

18. The machine learning apparatus of claim 17, wherein the first feature data and the second feature data are obtained by selecting a layer located in the same position in the plurality of first feature layers and the plurality of second feature layers, respectively.

19. The machine learning apparatus of claim 11, wherein the basic model is a previously learned machine learning model for image classification.

20. The machine learning apparatus of claim 11, wherein the basic model is a sub-machine learning model that is obtained through machine learning of a first training data set including the first analysis target data and the first analysis result data.
